(19) United States
(12) Patent Application Publication
(10) Pub. No.: US 2006/0127452 A1
(43) Pub. Date: Jun. 15, 2006

(54) Method and composition for reducing cravings for a craved substance

(76) Inventor: Lydia Muller, Phoenix, AZ (US)

Correspondence Address: SNELL & WILMER;ONE ARIZONA CENTER, 400 EAST VAN BUREN, PHOENIX, AZ, 850040001, US

(21) Appl. No.: 11/350,528

(22) Filed: Feb. 8, 2006

Related U.S. Application Data
(63) Continuation of application No. 10/345,657, filed on Jan. 16, 2003, now abandoned.
(60) Provisional application No. 60/417,582, filed on Oct. 10, 2002.

(51) Int. Cl. A61K 36/82; A61K 33/32; A61K 33/04; A61K 36/258; A61K 36/254; A61K 36/10; A61K 36/16; A61K 31/405; A61K 31/198

(52) U.S. Cl. 424/440; 424/728; 424/729; 424/762; 514/419; 514/563; 514/567; 514/184; 514/89; 424/641; 424/702; 424/752

(57) ABSTRACT

Disclosed is a composition and method for reducing cravings for a craved substance, particularly foods, with preparations containing 5-hydroxytryptophan (5-HTP). The 5-HTP is added to the same or similar substance that is actually craved to reduce the craving for the craved substance while further satisfying the craving by consumption of a reduced amount of the craved substance.

### CROSS REFERENCE TO RELATED APPLICATIONS

This application claims priority to, and the benefit of, U.S. provisional application 60/417,582, filed Oct. 10, 2002, which is hereby incorporated by reference.

### FIELD OF INVENTION

The present invention relates to methods and compositions for reducing cravings for a craved substance, and more particularly to compositions and methods for reducing cravings for foods, including the administration of a composition containing the active ingredient 5-hydroxytryptophan admixed with a carrier, wherein the carrier comprises at least part of the same or similar substance that is the subject of the craving.

### BACKGROUND OF INVENTION

A primary goal of substance abuse interdictions, such as with overeating, alcoholism, and drug abuse therapies, etc., is to reduce the craving urges for a craved substance by the substance abuser. Traditional treatment remedies offer drug formulations to reduce a patient's craving for a craved substance.

Alcohol abuse, for example, is often treated with tetraethylthiuram disulfide, more commonly known under the brand name ANTABUSE. This drug alters the metabolism of alcohol in the body, making it impossible for one who is taking the drug to drink without experiencing severe discomfort. When alcohol is present, the drug increases the concentration of acetaldehyde in the body, causing symptoms resembling those of a hangover, such that the patient feels hot, the face becomes flushed, the neck and head throb, and nausea, vomiting, and a headache may follow.

Cigarette smoking and/or nicotine addictions are commonly treated with nicotine replacement therapies. These therapies commonly employ other nicotine delivery modalities, such as gums, patches, nasal sprays, and inhalers to deliver smaller and smaller metered does of nicotine to allow the body to adjust gradually to sequentially smaller amounts of the craved substance. At the same time, the patient is encouraged to relearn daily behavioral patterns that have become associated with cigarette smoking, such as, for example, smoking after a meal or with a drink, or in response to stressful situations, and boredom.

Likewise, overeating is commonly treated by various methods including prescription drugs, over-the-counter drugs, nutritional supplements, and behavior modification therapies. These methods include use of appetite suppression formulations, such as, for example, DEXETRIM. Other methods, such as behavioral modification methods, seek to alter a patient's behavior by assigning points to various food types and limiting a patient's food intake according to this point system.

In general, various prior methods attempt to modify the habits of the patient by relying upon the patient's willpower to engage in a substitute behavior in response to a craving. For example, alcohol treatments rely on a patient's willpower to consume ANTUBUSE, drug therapies rely on the patient's willpower to ingest prescription drug alternatives, overeating therapies rely upon the patient's willpower to substitute a lower calorie food in response to a craving for a higher calorie food, in order to satisfy some predefined metric, such as, for example, a point system as described above.

Serotonin (5-HT) is thought to have a direct affect on various craving behaviors. For example, numerous studies demonstrate that medications affecting increases in brain serotonin are effective anorectic agents, which help obese patients lose weight and also decrease cravings for sweets and carbohydrates. Other studies have suggested that an increase in serotonin may also be effective at decreasing cravings for alcohol, nicotine, and cocaine, among others. 5-hydroxytryptophan (5-HTP) is thought to be an immediate precursor to serotonin. In this regard, it is believed that administration of 5-HTP leads to increased levels of serotonin, which in turn lead to a reduction in the various cravings described above. However, the effectiveness of 5-HTP has been limited, in part, by the problems described above; namely, the absence of formulations which motivate patients to consume 5-HTP, and, accordingly, reduce their consumption of a craved substance. Additionally, prior formulations of 5-HTP have lacked association with certain other important additives to effectively render a desired therapeutic benefit, such as metabolic enhancement, increased neurotransmitter production, mood elevation, increased energy, and other beneficial changes in one's physiological and emotional states, believed effective in facilitating changes in harmful behavioral patterns, that lead to overeating and/or over consumption of other craved substances.

Accordingly, a composition and method is needed to reduce a patient's consumption of a craved substance. Further, a composition and method is also needed to motivate a patient to consume a crave-reducing agent in response to a craving for a craved substance.

### SUMMARY OF THE INVENTION

This summary of the invention is intended to introduce the reader to various aspects and embodiments of the invention and is not a complete description of the invention. Particular aspects of the invention are pointed out in other sections hereinbelow, and the invention is set forth in the appended claims which alone demarcate its scope.

In accordance with one aspect of the present invention, a composition formulated to at least partially relieve cravings for a craved substance is provided. The composition includes a crave-reducing agent composed of 5-hydroxytryptophan. This novel composition may be useful in relieving cravings associated with various foods, especially foods higher in sugar content, various beverages, including alcoholic beverages, tobacco products as well as cravings for certain drugs, such as cocaine.

In accordance with a further aspect of the invention, a composition for facilitating weight loss is provided. As will be described in greater detail below, the composition includes various ingredients in combination with 5-hydroxytryptophan effective at reducing cravings and promoting weight loss. In various embodiments described hereinbelow, such ingredients include various chromium formulations, such as chromium piccoliate, in combination with a Vitamin B Complex, such as pyridoxyl-5-phosphate. In accordance with further exemplary embodiments, additional ingredients include gymnema sylvestre, ginger extract, green tea extract, garcinia cambogia, ginko biloba, cayenne pepper, cinnamon, cyanocobalmin, folic acid, Vitamin E, copper, amino acids, catalysts, co-factors, and the like.

In accordance with another aspect of the invention, a composition is provided wherein a crave-reducing agent is admixed into a carrier comprising a craved substance for consumption. The composition in accordance with this aspect of the invention promotes uptake of a crave-reducing agent. In one embodiment in accordance with this aspect of the invention, a crave-reducing agent including 5-hyroxytrptophan is admixed into a craved substance, such as, for example, hard candies, soft candies, chewable candies, and the like, for consumption. In accordance with this aspect of the invention, compositions and methods are provided to encourage uptake of a crave-reducing agent and decrease consumption of a craved substance by admixing the craving-reducing agent into a craved substance, or craved substance substitute, such as sugar-free candies, and the like.

### DETAILED DESCRIPTION

The following descriptions are of exemplary embodiments of the invention only, and are not intended to limit the scope, applicability, or configuration of the invention in any way. Rather, the following description is intended to provide convenient illustrations for implementing various embodiments of the invention. As will become apparent, various changes may be made in the function of compositional elements described in these embodiments without departing from the sprit and scope of the invention.

As used herein, “active ingredient,” “active substance” and/or “agent” includes any element, composition or material producing a beneficial effect, including vitamins, minerals, nucleic acids, amino acids, peptides, polypeptides, proteins, genes, mutagens, antiviral agents, antibacterial agents, anti-inflammatory agents, decongestants, histamines, anti-histamines, anti-allergens, allergy-relief substances, homeopathic substances, pharmaceutical substances, and the like.

As used herein, “craved substance” includes any food or drink-related substance, as well as any drug-related substance including any prescription, non-prescription, and/or homeopathic composition provided in any suitable delivery formulation including oral, intravenous, and inhalation administrations. In one embodiment, a craved substance may be any food-type substance, and further includes any sweetened-food substance such as baked goods including cookies, brownies, pies, cakes, and the like, and any candy-based substance including hard candy, soft candy, chewable candy, gums, and the like, and dairy-based products such as ice cream, yogurts, cheeses, chocolate milk, sweetened milk, and the like. As used herein, a food-type substance also includes any beverage-based substance including fruit and/or juice-based beverages, chocolate-based beverages including hot chocolate, alcoholic beverage, non-alcoholic beverage, carbonated beverages, sweetened beverages, non-sweetened beverages, and the like. In still further embodiments, the craved substance may be a drug-based substance including pain relievers, tranquilizers, depressants, sleep aids, tobacco substances, cocaine, marijuana, and the like.

As further used herein, “candy” and/or “confection” includes candies, cakes, and cookies of all types, includes the hard candies, lollipops, chewable candy, (taffy, caramel, tootsie rolls) chocolate candies (filled chocolate candies and unfilled chocolate candies), mint-type candies and gummy candies, and gums. Candy may include naturally sweetened candy, artificially sweetened candy and sweetened candy. As used herein, natural sweeteners include glucose, sucrose, fructose and the like, artificial sweeteners include aspartame, and others commonly known in the art.

As used herein, a “carrier” includes any of one or more components of the composition other than the active substance(s). In accordance with various embodiments of the invention, the carrier includes a craved substance and/or a substance that is analogous to the craved substance, such as any suitable food, beverage, and/or drug substitute.

In accordance with one aspect of the present invention, a composition formulated to at least partially relieve cravings for a craved substance is provided. The composition generally includes a crave-reducing agent and a suitable carrier. Suitable crave-reducing agents include 5-hydroxytryptophan, Biotin, and L-Glutamine. Preferably, the crave-reducing agent includes at least an effective amount of 5-hydroxytryptophan.

In accordance with another aspect of the present invention, a composition may be formulated to promote weight loss. The composition generally includes a crave-reducing agent in combination with at least one additional active ingredient effective at promoting weight loss. For example, such active ingredients may include appetite suppressants, thermogenic agents, metabolic agents, neurotransmitter production enhancement agents, mood enhancement agents, and the like.

In accordance with another aspect of the present invention, a composition may be formulated to promote mood enhancement and/or other cognitive processes and functioning. The composition includes a crave-reducing agent in combination with at least one additional active ingredient effective at promoting mood enhancement and/or cognitive processes.

In accordance with various embodiments of the present invention, the composition includes from about 75% to about 99.999% by weight of at least one carrier and an effective amount of an active substance, preferably a crave-reducing agent, in combination with at least one additional active substance. The effective amount of the active substance includes any amount of active substance required in a composition or dose suitable to render a beneficial therapeutic effect. For example, in accordance with one aspect of this embodiment, the composition includes from about 0.0000001% to about 5.0% by weight of at least one active substance, for example, 5-HTP. In a further example, the composition includes from about 25 mg to about 900 mg of 5-HTP per dosage, and more preferably from about 50 mg to about 150 mg of 5-HTP per dosage. In accordance with further exemplary embodiments, the composition additionally includes from about 200 mcg to about 400 mcg of GTF Chromium per dosage, and more preferably from about 100 mcg to about 200 mcg per dosage. In yet a further exemplary embodiment, the composition further includes from about 5 mg to about 200 mg of pyridoxyl-5-phosphate (“P5P”) per dosage, and more preferably from about 25 mg to about 50 mg per dosage.

In accordance with various other aspects and embodiments of the invention, various other ingredients, substances, and agents may be added to the composition to render various desired benefits. Exemplary additional components are further described herein below.

Crave Reducing Agents

As described above, suitable crave-reducing agents in accordance with the present invention include 5-HTP, Biotin, and L-Glutamine.

5-HTP is believed to act as a crave-reducing agent due to its role as a precursor of the neurotransmitter serotonin (5-HT). Studies have demonstrated that serotonin concentrations play an important role in eating behavior. A reduction in serotonin levels have been associated with increased food cravings, particularly sugar and carbohydrate cravings, and a corresponding increase in food consumption, whereas increased serotonin levels in patients are typically associated with a reduction in food cravings and food consumption. It is believed that additional intake of 5-HTP drives increased production of this important neurotransmitter, resulting in higher systemic levels of serotonin.

5-HTP may also act as an agent effective at promoting weight loss by encouraging patients to consume less of a craved substance. Studies have also demonstrated that 5-HTP promotes an earlier experience of “satiety” or fullness. Accordingly, patients consuming a composition including 5-HTP in accordance with the present invention may be motivated to eat less of a particular craved substance due to an earlier feeling of satiety or fullness.

In addition to promoting crave reductions and weight loss, 5-HTP is also believed to act as a mood-elevating agent. Low levels of serotonin have been associated with a variety of health-related issues including depression, anxiety, compulsive behaviors including overeating, substance abuse, and the like. In accordance with the present invention, it is believed that 5-HTP alone, and particularly in combination with additional active ingredients, renders a therapeutically beneficial benefit in treatment of one or more of these conditions.

As a mood elevator, 5-HTP is believed to promote feelings of calm and satisfaction. In this regard, it is also believed that 5-HTP helps alleviate compulsive behaviors of various types, including overeating and substance abuse, because patients undergoing administration of 5-HTP are less likely to consume substances of various types to promote emotional wellbeing.

L-Glutamine is also an effective agent in accordance with the present invention at reducing cravings for sugars and alcohol, among others. L-Glutamine is an amino acid which is believed to readily pass through the blood-brain barrier where it is converted to glutamic acid, which is further believed to reduce fatigue. In accordance with various embodiments the composition includes from about 10 mg to about 500 mg of L-Glutamine per dosage.

Biotin is also an effective agent in accordance with the present invention at reducing cravings for sugars, among others.

Appetite Suppressing Agents

L-Tyrosine is also an effective agent that may be used in accordance with the present invention. L-Tyrosine is an amino acid and a precursor to neurotransmitters dopamine and nor-epinephrine. It is believed to act as an appetite suppressant, elevate one's mood, reduce fatigue, promote alertness and memory and otherwise enhance cognitive functioning, as well as facilitating adrenal, pituitary, and thyroid regulation. In accordance with various embodiments, the composition includes from about 10 mg to about 500 mg of L-Tyrosine per dosage.

Garcinia Cambogia is also an effective agent that may be used in accordance with the present invention. Among others, Garcinia Cambogia is useful as an appetite suppressant. In accordance with various embodiments, the composition includes from about 10 mg to about 500 mg of Garcinia Cambogia per dosage.

Thermogenic Agents

Chromium is an effective agent useful in accordance with the present invention, particularly as a thermogenic agent. In general, thermogenic agents are thought to promote an increase in metabolic processes by, among other things, effecting a slight increase in a patient's basal temperature. Chromium is also believed to be an important co-factor in regulating insulin, which, among other things, affects the metabolism of blood sugar into stored fat.

DHEA (dehydroepiandrosterone) is also useful as a thermogenic agent in accordance with the present invention. DHEA is also useful as a stimulant, reducing fatigue, and improving cognitive functions. In an alternative embodiment, 7-Keto DHEA, a more active form of DHEA, may be used instead. In accordance with various embodiments, the composition includes from about 10 mg to about 100 mg of DHEA per dosage.

Cayenne pepper may also be utilized as a thermogenic agent. Among other things, cayenne pepper is recognized at inducing body fat mobilization and utilization, promoting blood flow, and promoting an invigorating effect on the body. In accordance with various embodiments, the composition includes from about 10 mg to about 100 mg of cayenne pepper per dosage. Ginger may also be utilized as a thermogenic agent. Ginger is thought to be useful at counteracting nausea, particularly nausea that may be induced by 5-HTP in some patients. In accordance with various embodiments of the present invention, the composition includes from about 10 mg to about 50 mg of ginger per dosage.

Neurotransmitter Production Agents

In accordance with the present invention, enhanced neurotransmitter production is believed to be important for promoting mood elevation and overall wellbeing. As further discussed above, certain conditions and behaviors, particularly compulsive behaviors such as overeating and substance abuse, are believed to be mitigated or avoided where the patient, because of a balanced and healthy mental state, is not motivated to turn to such harmful substances and behaviors to alter his or her mental state.

Vitamin B complex, vitamin B-12 and folic acid may be used in accordance with the present invention to facilitate neurotransmitter production. Vitamin B complex is believed to be a co-factor involved in neurotransmitter production. Additionally, it is also believed to be involved in the production of energy and have a mild antidepressant effect.

Green tea may also be used in accordance with the present invention. Green tea contains catechins and caffeine, among others. These substances are particularly useful in maintaining norepinepherine levels in the brain and are further believed to inhibit enzymatic breakdown of norepinepherine. In accordance with various embodiments of the present invention, the composition includes from about 20 mg to about 250 mg of green tea extract per dosage.

Additional Ingredients

In accordance with various aspects and embodiments of the invention, 5-HTP may be obtained from any suitable source, including a plant extract, such as Griffonia Simplicifolia extract, commercially available from Kaden Biochemicals, Inc., 17, Camden Road, Belle Mead, N.J. 08502. Certain other ingredients are preferably added to the composition in accordance with various aspects and embodiments of the present invention. These include the following: Vitamin E, which is believed to increase HDL cholesterol levels and aid in fatty acid metabolism, as well as act as an antioxidant; Zinc, which is believed to aid in promoting insulin potency and efficiency as well as increasing a patient's glucose tolerance; Vitamin C and Manganese, both of which are further believed to promote insulin potency; Vanadium and Vanadyl Sulfate, both of which are believed to promote glucose tolerance; Coenzyme Q 10, also known as “Ubiquinone” and Ginseng are believed to enhance energy production and utilization, thereby reducing fatigue as well as promoting cognitive functioning; likewise Copper is also believed to be a co-factor responsible for energy production, and may be provided at a dosage range from about 0.5 mg to about 3 mg; Cinnamon is believed to stimulate metabolic processes, and may be provided at a dosage range from about 1 mg to about 100 mg; Gymnema Sylvestre is believed to block the absorption of sugar as well as increase the activity of insulin and may be provided at a dosage range from about 50 mg to about 500 mg; Pantothenic Acid is believed to support healthy adrenal gland functioning; and Ginko Biloba is believed to increase blood flow to the brain, as well as increasing the number of serotonin receptors in the brain, and may be provided at a dosage range from about 25 mg to about 50 mg.

In accordance with another aspect of the present invention, a composition and method is provided to motivate a patient to consume a crave-reducing agent in response to a craving for a craved substance. The method includes the step of providing a composition wherein a crave-reducing agent, preferably 5-HTP, is provided in a carrier that includes the substance that the patient craves. For example, in one embodiment, 5-HTP is provided in a craved food substance, particularly candy, for administration to patients craving carbohydrates, particularly sugars. In another embodiment, 5-HTP is provided in a composition containing alcohol, such as an alcoholic beverage. In another embodiment, 5-HTP is provided in a composition containing cocaine, wherein the composition is configured for administration to the patient by any suitable method including oral and nasal administration. In another embodiment, 5-HTP is provided in a composition containing nicotine, wherein the composition is configured for administration by any suitable method including transdermal administration.

In accordance with this invention, a patient is provided with a craved substance in combination with 5-HTP. In this regard, the patient's immediate need for gratification is addressed by providing immediate access to the craved substance, while at the same time promoting intake of 5-HTP to reduce the cravings for the substance craved. In this regard, the patient is able to modify physiological responses to cravings over time while still addressing the emotional/behavior needs to consume at least some amounts of the craved substance while such modifications are taking place.

### EXAMPLES

Examples set forth hereinbelow are illustrative of various aspects of certain preferred embodiments of the present invention. The compositions, methods and various parameters reflected therein are intended only to exemplify various aspects and embodiments of the invention, and are not intended to limit the scope of the claimed invention.

### Example #1

Formula

### Lollipop

Combine 5 g Griffonia simplicifolia extract, 2.5 g Pyridoxyl-5-phosphate, 5 g Green Tea extract, 10 g Gymnema sylvestre extract, 10 g Tyrosine, 2.4 g Stevia extract, 100 mg
Chromium polynicotinate, and 10 g L-Glutamine, mixing powders thoroughly in a lab blender.
Add above powder mixture and 16 ml natural flavor concentrate to Hystar brand lollipop base (included but not limited to) heated to approximately 200 degrees Fahrenheit.
Mix thoroughly.
Pour into prepared, lubricated mold.
Insert sticks.
Allow to solidify.
Remove from mold. Wrap individually. Place in childproof packaging.
   Final Product:
Each 8 g Lollipop contains:
100 mg Griffonia simplicifolia extract 98%.
200 mcg Chromium polynicotinate GTF
100 mg Green Tea extract
100 mg Gymnema sylvestre extract
100 mg L-Tyrosine
100 mg L-Glutamine
25 mg Pyridoxyl-5-phosphate

### Example #2

### Caramels

Combine 5 g Griffonia simplicifolia extract, 2.5 g Pyridoxyl-5-phosphate, 5 g Chromium polynicotinate, 5 g Ascorbyl palmitate, mixing powders thoroughly in a lab blender.
Add powder mixture to 500 g lycasin (including but not limited to) caramel candy base heated to 200 degrees Fahrenheit.
Mix thoroughly.
Add 5 ml flavor concentrate (flavor optional) and mix thoroughly.
Pour into prepared, lubricated mold.
Allow to cool/set until solidified.
Remove from mold. Wrap individually. Place in childproof packaging.
   Final Product:
Each 8 g lollipop contains:
50 mg Griffonia simplicifolia extract 98%
100 mcg Chromium polynicotinnate GTF
25 mg Pyridoxyl-5-phosphate
50 mg Ascorbyl palmitate

### Example #3

### Chocolate Meltaway Confection

Combine 7.5 g Griffonia simplicifolia extract, 2.5 g Pyridoxyl-5-phosphate, 100 mg Chromium polynicotinate, 2.5 g Ginger extract, mixing powders thoroughly in a lab blender.
Heat 400 g Maltitiol proprietary chocolate confection base (included but not limited to) to approximately 200 degrees Fahrenheit.
Add 25 ml 95% Ethanol to the above powder mixture to dissolve ingredients.
Add the above solution to the candy base and mix to uniformly disperse ingredients.
Pour into prepared molds. Wrap individually. Place in childproof packaging.
   Final Product:
Each 4 g confection contains:
75 mg Griffonia Simplicifolia extract 98%
100 mcg Chromium polynicotinate GTF
25 mg Pyridoxyl-5-phosphate
25 mg Ginger extract

The present invention has been described above with reference to a number of exemplary embodiments and compositional elements. It should be appreciated that the particular embodiments shown and described herein are illustrative only and are not intended to limit in any way the scope of the invention as set forth in the claims. Those skilled in the art having read this disclosure will recognize that changes and modifications may be made to the exemplary embodiments and various constituent elements without departing from the scope of the present invention. For example, although reference has been made throughout to 5-HTP as a crave-reducing agent, it is intended that the invention also be applicable to any suitable crave-reducing agent that may be suitably combined for use with a craved substance. Further, although various constituent elements are set forth with exemplary compositional ranges, such as by weight percent or dosage, practitioners will appreciate that any suitable range of any suitable element, listed herein, or otherwise, may be used without departing from the scope of the invention. Further, although certain preferred aspects of the invention are described herein, such as lollipops, caramels, and confections, for example, practitioners will appreciate that a crave-reducing agent may be admixed with any suitable craved substance, not limited to those herein described, without departing from the scope of the invention. Accordingly, these and other changes or modifications are intended to be included within the scope of the present invention, as expressed in the following claims.

1. A composition for reducing a craving for a craved substance, the composition comprising:
a carrier; and
and a crave-reducing agent comprising 5-hydroxytryptophan,
wherein the carrier comprises properties that are the same or similar to the craved substance.

2. A composition for reducing a craving for a craved sugar containing substance to promote weight loss, the composition comprising:
a carrier containing at least one of a candy and a confection; and
a weight-reducing agent,
wherein the weight-reducing agent includes a crave-reducing agent and an appetite-suppressing agent.

3. The composition of claim 2, wherein the crave-reducing agent includes at least one of the following: 5-hydroxytryptophan, L-Glutamine, and biotin; and wherein the appetite-suppressing agent includes at least one of the following: L-Tyrosine, and Garcinia Cambogia.

4. The composition of claim 3, wherein the weight-reducing agent further comprises a thermogenic agent comprised of at least one of the following: chromium, dehydroepiandrosterone (DHEA), and cayenne pepper.

5. The composition of claim 4, wherein the weight-reducing agent further comprises at least one of the following: Cinnamon, Copper, Coenzyme Q, Gingko biloba, Ginseng, Gymnema Sylvestre, Manganese, Pantothenic Acid, Vanadium, Vanadyl Sulfate, Vitamin C, Vitamin E, Zinc.

6. A lollipop composition for reducing a craving for a craved substance to promote weight loss, the lollipop composition comprising:
a carrier comprising a lollipop base, and
from about 25 to about 150 mg 5-hydroxytryptophan.

7. The lollipop composition of claim 6, further comprising:
from about 50 to about 400 mcg chromium polynicotinate, and
from about 5 mg to about 50 mg pyridoxyl-5-phospate.

8. The lollipop composition of claim 7, further comprising
from about 20 mg to about 250 mg of green tea extract;
from about 50 mg to about 500 mg of Gymnema Sylvestre extract;
from about 10 mg to about 500 mg of L-Tyrosine; and
from about 10 mg to about 500 mg of L-Glutamine.

9. A method for reducing a craving for a craved substance, the method comprising the steps of:
providing a crave-reducing composition including a crave-reducing agent wherein the crave-reducing agent includes 5-hydroxytryptophan, and
admixing the crave-reducing agent into a carrier, comprising at least one of a food-based substance and a drink based substance selected from the group consisting of: cookies, brownies, cakes, hard candies, soft candies, chewable candies, gums, ice creams, yogurts, milk shakes, juice-based beverages, chocolate-based beverages, carbonated beverages, and mixtures thereof.

10. (canceled)

11. (canceled)

12. (canceled)

13. A caramel composition for reducing a craving for a craved substance, the caramel composition comprising:
a carrier comprising a caramel base; and
from about 25 mg to about 150 mg of 5-hydroxytryptophan;
from about 50 mcg to about 400 mcg of chromium polynicotinate; and
from about 5 mg to about 50 mg of pyridoxyl-5-phospate.

14. A chocolate composition for reducing a craving for a craved substance, the chocolate composition comprising:
a carrier base comprising a chocolate composition; and
from about 50 mg to about 150 mg of 5-hydroxytryptophan;
from about 50 mcg to about 400 mcg of chromium polynicotinate; and
from about 5 mg to about 50 mg of pyridoxyl-5-phospate.
